(19) United States
(12) Patent Application Publication
(10) Pub. No.: US 2009/0093712 A1
(43) Pub. Date: Apr. 9, 2009

(54) Method and device for navigating a catheter through a blockage region in a vessel

(75) Inventors: Erik Busch, Hemhofen (DE); Klaus Klingenbeck-Regn, Nurnberg (DE); Johann Uebler, Nurnberg (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 12/286,676

(22) Filed: Oct. 1, 2008

Related U.S. Application Data
(60) Provisional application No. 60/977,741, filed on Oct. 5, 2007.
(60) Provisional application No. 61/036,546, filed on Mar. 14, 2008.

(51) Int. Cl. A61B 17/22; A61B 6/03

(52) U.S. Cl. 600/424; 606/159; 128/898

(57) ABSTRACT

The invention relates to a method for navigating a catheter with a catheter tip through a blockage region in a vessel, especially a coronary vessel, whereby the catheter is pushed forward under real-time radiological observation. The underlying objective of the invention is to arrange such a method in such a way that it permits especially simple, rapid and low risk navigation of a catheter through the blockage region in the vessel. For this purpose, in accordance with the invention a three-dimensional path through the blockage region is determined by reference to a set of sectional images or a 3D representation of the blockage region, recorded beforehand as part of a preliminary investigation, whereby a data set including the path coordinates is brought into register with the real-time radiological images, and whereby the path or a projection of the path is visualized on a display, overlaid on the real-time radiological images.

A clean copy of the abstract that incorporates the above amendments is provided herewith on a separate page.

### CROSS REFERENCE TO RELATED APPLICATIONS

The present application claims the benefit of a provisional patent application filed on Oct. 5, 2007, and assigned application No. 60/977,741. The present application also claims the benefit of another provisional patent application filed on Mar. 14, 2008, and assigned application No. 61/036,546. Both of the applications are incorporated by reference herein in their entirety.

### FIELD OF THE INVENTION

The invention relates to a method for navigating a catheter with a catheter tip through a blockage region in a vessel, in particular a body vessel, especially a coronary vessel, whereby the catheter is pushed forward under real-time radiological observation. The invention relates in addition to an associated medical investigation and treatment device.

### BACKGROUND OF THE INVENTION

Heart infarcts are among the conditions which most frequently result in death. In approximately 20% to 30% of all documented cases, a heart infarct occurs as a result of chronically blocked or closed up coronary vessels. The total closure of a coronary artery over a period of more than 30 days is referred to as a “chronic total occlusion” (CTO). Earlier, such conditions were mostly treated with medication or in serious cases by a bypass operation on the open heart. In more recent times, minimally invasive interventions with a catheter, which is brought into position in the region of interest for investigation and treatment through the patients bloodstream (so-called percutaneous intervention), have established themselves as promising alternatives. The objective of such an intervention could be, for example, to achieve the opening of a blocked or stenosed section of a vessel followed by its widening with an expandable dilatation balloon, during which if necessary a stent is also implanted to permanently support and hold open the section of vessel which has been widened. However, provision can also be made to undertake, prepare or plan a purely diagnostic intervention, e.g. the introduction of a catheter equipped with imaging sensors or physiological sensors.

However, it is precisely in the case of a totally blocked vessel that a catheter-based intervention is extremely time-consuming, difficult and risky, because prior to the balloon dilation it is first necessary to break through the section of the vessel which is closed up by calcium deposits, using the catheter tip or the guide wire provided to guide the catheter, as appropriate. In doing this there is a substantial danger that when it is being advanced the catheter tip or the guide wire, as applicable, is pushed off course sideways or radially outwards at centers of hardness or the like, and pierces the wall of the vessel, which can lead to severe internal bleeding and other complications. Many sites can also be hardened in such a way that they can only with difficulty be broken through.

Usually, the breakthrough of the blockage region using the catheter tip, which prepares for the actual (later) intervention, is effected under radiological or angiographic X-ray control. In doing so, the regions of the vessels which are of interest are imaged “live” by the injection of an X-ray contrast agent, and X-ray projection images are made visible by showing them on a display (X-ray through-illumination, also referred to fluoroscopy). However, as the blood flow in the affected vessel is blocked by the total blockage, the contrast agent can never propagate beyond the boundary surface of the blockage site. The blockage site itself is thus not visible in the fluoroscopic image. The doctor or medic performing the treatment is thus essentially reliant on his tactile awareness when advancing the catheter tip through the blockage region, which makes the procedure very difficult and high risk.

### SUMMARY OF THE INVENTION

It is therefore the objective of the invention to specify a method which permits—in particular for the preparation or planning of a later diagnostic, surgical or therapeutic intervention—especially simple, rapid and low risk navigation of a catheter through a blocked region of a vessel, in particular a coronary vessel. In addition, it is to specify a medical investigation and treatment device which is particularly suitable for this purpose.

In respect of the method, the objective is achieved in accordance with the invention in that a three-dimensional path through the blockage region is determined by reference to a set of sectional images or a 3D representation of the blockage region, recorded or reconstructed beforehand as part of a preliminary investigation, whereby a data set including the path coordinates is brought into register with the real-time radiological images, and whereby the path or a projection of the path is visualized on a display, overlaid on the real-time radiological images.

Underlying the invention is a recognition that the blocking plugs for completely blocked vessels generally contain three main types of deposit (plaque), namely:
1. hard plaque comprising calcium deposits,
2. fibrous plaque (fibrous connective tissues),
3. soft plaque (predominantly blood clots and soft tissue with a high fat content)

Structures of this type can be comparatively well identified and spatially delimited from each other, with high resolution of soft tissues, in imaging methods which generate sectional images, e.g. by computer tomography, 3D angiography, magnetic resonance tomography, 3D ultrasound imaging, positron emission tomography or single photon emission tomography.

The invention starts from the idea of collecting data of this type about the structure and composition of blockages in vessels as part of a preliminary investigation and then using it for the selective control or navigation of a catheter through to the other side of the blockage region. In doing this, the catheter should as far as possible be guided so that the catheter tip (or tip of the guide wire, as applicable) preferably cuts through the especially soft areas of the vessel cross-section, in particular the soft plaque or especially soft blood clots.

To this end, provision is expediently made to map the blocked section of vessel, as part of the preliminary investigation, by a plurality of sectional images lying essentially at right angles to the axis of the vessel (i.e. perpendicular to the main direction of advance of the catheter) and in each of the sectional images to identify the especially soft places, in particular the softest ones. This will preferably take place automatically in a computer-aided analysis unit, with the help of methods of medical image and pattern recognition which are familiar to the specialist. To this end, in the case for example of X-ray based imaging methods, each section of the image will have assigned to it as appropriate the tissue, or one of the three classes of tissue mentioned above, which is present in it, by reference to its Houndsfield number or a similar characteristic value, using empirically or theoretically known relationships and assignment tables. In the case of tomographic images based on magnetic resonance methods it is possible, for example, to use the water content of the region concerned, which can be deduced from the images, as the basis for assigning a tissue.

After possible breakthrough points or areas for the later advance of the catheter have been identified in this way in each of the sectional images, this is followed by a search, using optimization methods which are also known in principle, for as optimal a path as possible, or “path of least resistance” (PLR), through the blockage region, or possibly even a sheaf of such paths. This will also preferably take place fully automatically, using computer-implemented algorithms. In the simplest case, the initial step in doing so will be to identify a breakthrough point in each of the sectional images of the blockage region recorded in the preliminary investigation. The breakthrough points are then linked together by spatial interpolation to form a spatial path (=curve in space). More complicated cases, in which the regions of soft plaque are not contiguous, can in principle also be managed with appropriate adaptation of the method. In this way, even before insertion of the catheter, a route or path through the blockage region in the vessel is defined, along which it will be possible to push the catheter forward with the least possible mechanical resistance. The optimal path determined in this way forms the intended plan, as it were, for the actual forward movement of the catheter.

It is also possible, rather than specifying exactly (point-by-point) the path to be followed, to define essentially only the enclosing surface of a spatial region of soft plaque as the outer boundary of the sheaf of possible paths.

In addition, in selecting a particularly suitable type of catheter or guide wire, in particular with appropriate flexibility or stiffness, reference can advantageously be made to the data derived from the preliminary investigation, about the nature, composition and spatial arrangement of the plaque in the blocked section of vessel.

The insertion of the catheter into the vessel concerned and the breaking through of the blockage region by the catheter tip is then advantageously effected under angiographic X-ray control. As is known per se from the prior art, when this is being done the regions of the vessel which are of interest are made visible in the X-ray through-illumination by the injection of an X-ray contrast medium and are shown “live” on a display in the X-ray system.

For improved control and guidance of the catheter tip through the blockage regions, which in the X-ray projection image is shown in structureless form (see above), under the concept presented here a representation of the “optimal” path (PLR) is overlaid on the X-ray projection image on the display. The reconciliation which this requires, of the relevant coordinate systems, namely the coordinate system for the optimal path on the one hand and the coordinate system for the current real-time X-ray image on the other hand, is preferably effected with computer assistance in real time using methods of 2D or 3D registration and image merging which are known per se.

The result is that, for example, a line which is highlighted in color is overlaid on the live X-ray projection image on the display, representing the optimal path for the catheter tip to follow as it advances, projected in the correct position in this image. As the current position of the catheter tip is generally also easy to see in the X-ray image, at any rate if appropriate X-ray markers are attached, the person monitoring the catheter advance or the supervising doctor or therapist can easily detect visually any deviations from the planned path, and correct them as the advance proceeds.

In addition or as an alternative to X-ray opaque markers in the region of the catheter tip, electrical or electromagnetic position sensors or the like can also be attached there, by reference to which it is possible to determine the current position and/or orientation of the catheter tip in the vessel so that it can be marked—after any necessary reconciliation of the coordinates or after a suitable coordinate transformation—in the correct position in the real-time angiographic image, e.g. by highlighting in color.

In particular in the case that the catheter advance is effected manually or semi-automatically, it can be logical to indicate in addition on the display, in the form of a color-coded scale or suchlike, a value characteristic of the mechanical resistance to be expected as the catheter is advanced, derived from the sectional images or the 3D representation of the blockage region or, as appropriate, from the assigned tissue and its characteristics. This gives the medic carrying out the procedure a useful piece of additional information, so that he/she can be well prepared for the tactile conditions which are to be expected as the catheter advance proceeds.

In an embodiment, alternative or additional to that described above of an overlay display of the real-time angiographic image with the path of least resistance merged onto or overlaid on it, it is advantageous if the display shows, apart from the real-time angiographic image, a sectional image of the blockage region, corresponding to the current position of advance of the catheter tip in the bodily vessel, recorded during the preliminary investigation. In this way, what might be called a “virtual endoscope” is realized, so that the person carrying out the intervention or the supervising medic sees in front of them on the display a sectional view of the plaque lying in front of the catheter tip, as though the catheter itself were equipped with an appropriate imaging sensor for in-situ imaging. The sectional views shown can then be suitably presented, e.g. by colored highlighting of the areas of the sectional surfaces identified by the analysis algorithm as particularly soft.

With this type of representation is it useful in addition if the breakthrough point of the path which has been determined is marked on the sectional view displayed. Over and above this it is advantageous for a comparison of the planned and actual positions if the current spatial position of the catheter tip is detected, e.g. with the help of position sensors or position detectors attached to the catheter, and is marked on the sectional image displayed.

In a particularly advantageous variant, the catheter tip is automatically kept on the path of least resistance which has been determined. To this end, it is expedient if the current spatial position of the catheter tip is detected with the help of position sensors and is compared against the coordinates of the path. Using the deviation determined—e.g. relative to the sectional plane corresponding to the catheter tip's current position of advance—and by reference to the further course (directional vector) of the optimal path previously defined, suitable control instructions are calculated to keep the catheter tip on the optimal path or return it to that path during its subsequent advance. It is advantageous if the control signals which are calculated are communicated to a steering and drive device which effects the directed advance of the catheter, thus realizing fully-automated guidance of the catheter or the guide wire, as applicable, along the desired path.

Preferably, the guidance provided will be magnetic, where the (magnetic) catheter tip is steered by externally applied magnetic fields.

Alternatively or additionally, a manual or semi-automatic mode of operation can also be provided. In the case of the manual mode of operation, the medic performing the treatment is, as hitherto, reliant his/her tactile senses during the advance of the catheter, but does have available on the display the additional data about the position and orientation of the catheter tip and/or about the internal structure and thickness of the vessel blockage. In the semi-automatic mode, for example, a motorized drive or motor-driven directional steering of the catheter tip is indeed provided, but this is actuated by a manual operating interaction, e.g. in that the doctor performing the treatment uses a computer mouse or another suitable data input device to move a mouse cursor or the like on the display showing the graphic object, such as a reconstructed 3D model of the blockage region. In both the manual and the semi-automatic modes it is possible, if required, to merge onto the display various types of navigation aids, e.g. directional arrows, red and/or green signal lights (“stop/go/care!”) and the like.

It is advantageous if there is also a possibility for recalculating or interactively modifying a previously defined path through the blockage region if this is called for by medical facts newly ascertained in the course of the intervention or by the progress of the intervention itself, e.g. by unanticipated deviations (dynamic path adjustment).

It is, furthermore, advantageous if data about the blockage region, about the path which has been determined through the blockage region, about the actual course of the movement as the catheter advances, about the progress over time of the procedures and/or about the medical and technical equipment used, is communicated to a database of an associated medical expert system and is stored there for later “self-learning” analysis, preferably based on artificial intelligence methods. The algorithms for determining the optimal path can thus be systematically improved and optimized by the empirical knowledge accumulated over the course of time. For this purpose, the medical investigation and treatment system which includes the catheter will either itself be equipped with suitable electronic modules, or will have suitable interfaces for a connection to an appropriate external expert system, possibly networked with other expert systems.

In respect of the device, the objective mentioned in the introduction is achieved by a medical investigation and treatment device incorporating
a catheter which can be introduced into a bodily vessel with a blockage region,
an angiographic imaging system for real-time monitoring of the catheter advance through the blockage region,
an electronic planning unit, for planning the catheter advance, which is configured in such a way that it determines a three-dimensional path through the blockage region by reference to a set of sectional images or a 3D representation of the blockage region previously recorded as part of the preliminary investigation,
an image registration and overlay unit, which is configured in such a way that it registers and overlays a data set incorporating the path coordinates against the real-time angiographic images,
together with a display unit with a display on which the real-time angiographic images are displayed showing the path as an overlay.

The advantages which the invention aims to achieve consist particularly in making available to the doctor or medic performing a procedure, by the appropriate processing and visual display of medical (image) data obtained as part of a preliminary investigation, an effective navigation aid in the planning and performance of a catheter insertion into chronically blocked vessels, which relieves the burden on them and reduces the demands on their tactile skills and their experience. Automated, reproducible definition of the “path of least resistance” restricts the latitude for human decision-making, to the benefit of the medic performing the procedure, in precisely those situations in which the susceptibility to incorrect decisions, with the associated risks, is especially high. CTO treatments can thus be performed with reduced risk, in a shorter time than hitherto, as a result of which in particular the patient's exposure to radiation (from the X-ray illumination which accompanies the intervention) can also be kept low.

Even if the invention has here been described in a medical context, the specialist will nevertheless recognize that its basic idea could also be directly realized for other catheter-aided investigations, e.g. in the investigation of blockage sites in sewage pipes or the like, provided that the imaging procedure is appropriately modified with respect to the typical material parameters etc. for the object under investigation.

### BRIEF DESCRIPTION OF THE DRAWINGS

An exemplary embodiment of the invention is explained in more detail by reference to a drawing. This shows, in each case as a highly simplified and schematic representation:

FIG. 1 a longitudinal section through a vessel with a blockage region, where several sectional planes are marked, together with a path through the blockage region, by appropriate symbols,

FIG. 2 a cross-section through the blockage region in the vessel shown in FIG. 1,

FIG. 3 a sketch of the principle of a medical investigation and treatment device with a catheter to be introduced into a vessel,

FIG. 4 a flow diagram to illustrate sequences of activities during the operation of the investigation and treatment device shown in FIG. 3, and

FIG. 5 another flow diagram, to illustrate a sequence of activities which differs from that in FIG. 4.

### DETAILED DESCRIPTION OF THE INVENTION

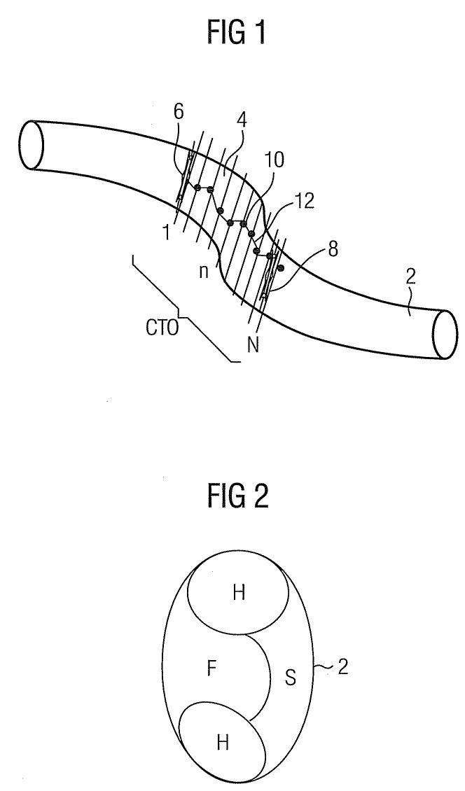

FIG. 1 shows a highly schematic view of a longitudinal section through a section of a vessel 2, here a coronary artery, which in a blockage region 4 has a total blockage (CTO=chronic total occlusion), caused by calcium and other deposits. To prepare for a catheter intervention, in which the blockage region 4 is to be broken through by the catheter tip of a catheter introduced into the vessel, a series of computer tomography sectional images of the blockage region 4 is first recorded as part of a preliminary investigation. The sectional planes of the individual sectional images—here N of them, starting with sectional plane 1 at the proximal end 6 of the blockage region 4 through to sectional plane N at the distal end 8—are so aligned that each of them shows a cross-section through the essentially cylindrical vessel 2. The vessel 2 is thus, as it were, virtually cut into numerous slices in the region of the blockage.

Such a cross-sectional image typically discloses an inner structure to the blockage of the vessel, roughly as shown in FIG. 2. On the one hand there are regions of hard plaque H, of coronary calcium deposits. On the other hand there are regions of fibrous plaque F. Finally, there are generally also other regions of soft plaque S. Regions of this last type, with soft plaque S, are especially well suited for piercing with a catheter tip.

For the purpose of planning the piercing by a catheter through the blockage region 4 therefore what might be called an “optimal itinerary” is compiled, so that as far as possible the catheter tip crosses exclusively through the areas with soft plaque S. In the simplest case of a volume of soft plaque S which is contiguous from the proximal end 6 through to the distal end 8 then, for example, a breakthrough point 10 is defined for the catheter tip in each sectional image, through the soft plaque S within the sectional area, roughly in a central region of this area, and the breakthrough points 10 so defined are imagined to be joined together from one sectional image to the next. As the result of this procedure, one obtains a three-dimensional spatial curve or a path 12 through the blockage region 4, the so-called “path of least resistance” (PLR), which is drawn in on FIG. 1 (as a two-dimensional projection).

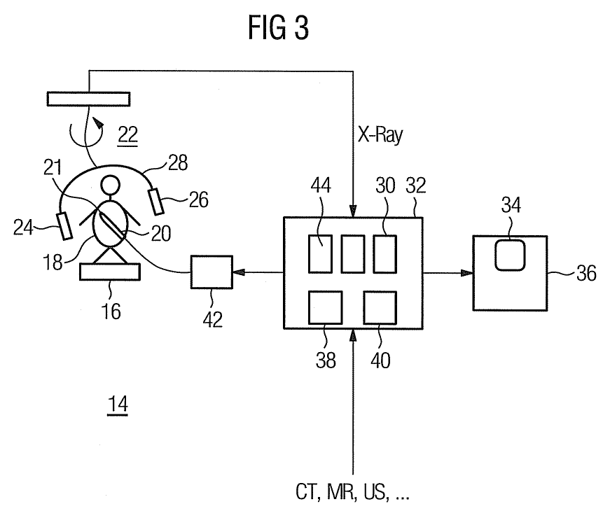

FIG. 3 shows schematically a medical investigation and treatment device 14, in which the concept of the path of least resistance, presented in simplified form above, is applied. The investigation and treatment device 14 incorporates a examination table 16, on which a patient 18 can be placed. A catheter 20, with a catheter tip 21 arranged on the end which is in the body, is introduced into the patient's blood stream and is advanced as far as the blockage region 4 of the coronary artery concerned.

The catheter advance is effected under angiographic X-ray control. For this purpose, an angiographic X-ray system 22 is provided, with a radiation source 24 and a detector 26 which are mounted opposite each other on a C-Arm 28 and can be rotated around the patient 18. An assigned image processing unit 30, which is part of a control and processing unit 32 shown here only schematically, generates, from the signals captured by the detector 26, X-ray projection images of the blocked region of the vessel, which can be shown on a display 34 in a display unit 36.

The control and processing device 32 is in addition connected via suitable interfaces to a medical image database (not shown) in which are stored the CT or other sectional images of the blocked region 4 of the vessel 2 concerned, recorded as part of the preliminary investigation. These sectional images or the associated datasets, as applicable, which can also for example be based on ultrasound or magnetic resonance imaging, are loaded into a planning unit 38 in the control and processing device 32 which, on the basis of computer-implemented analysis and optimization algorithms, determines the path 12 of least resistance, in the sense described above, through the blockage region 4 and provides it for subsequent use in the form of a dataset of spatial coordinates.

In an image registration and overlay unit 40 in the control and processing device 32, this dataset is combined, correctly positioned, with the angiographic image data which is recorded “live”, so that the path 12 which has been determined can be shown as a suitable projection overlaid on the real time angiographic images on the display 34. Also shown on the display 36, apart from the real-time angiographic image, is a sectional image of the blockage region 4 corresponding to the current point of advance of the catheter tip 21 in the vessel 2, recorded beforehand during the preliminary investigation, on which are marked both the breakthrough point 10 on the path 12 which has been determined and the current position of the catheter tip 21. The position and orientation of the catheter tip 21 are here known from position sensors attached to the catheter 20.

The advance and steering of the catheter through the blockage region 4 of the vessel 2 along the path 12 of least resistance which has been determined will preferably be effected fully automatically. For this purpose, the catheter 20 is provided with a drive and steering device 42, which is either motor driven or can be controlled by variable external magnetic fields, and which receives appropriate control signals from a targeting guidance unit 44 in the control and processing device 32. In this targeting guidance unit 44, computer-implemented algorithms are used to compare the current position of the catheter tip 21, reported by the position sensors, against the planned data based on the path 12 of least resistance and a corresponding signal to the drive and steering device 42 is calculated, to control or correct the subsequent advance, as appropriate.

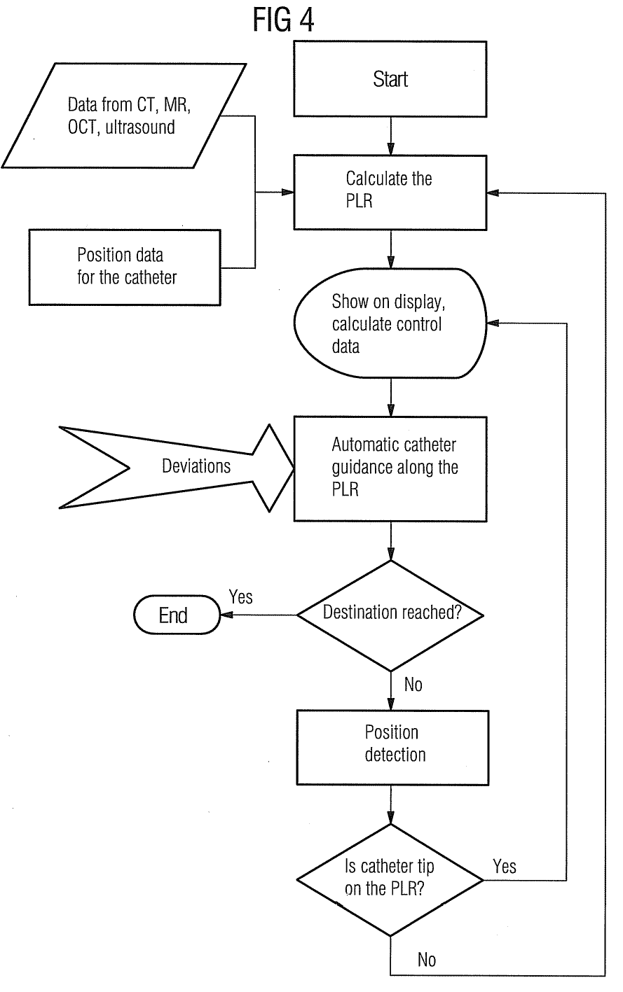

That is to say, in this fully automatic mode of operation the principle of a closed feedback loop is applied to keep the catheter tip 21 on the path 12 which has been determined, this being illustrated once again in diagrammatic form in the flow diagram shown in FIG. 4. In doing so, it is also possible to effect if necessary a dynamic iterative adaptation and recalculation of the path 12, depending on the current position of the catheter tip 21, this being manifest in the path loops shown in the flow diagram.

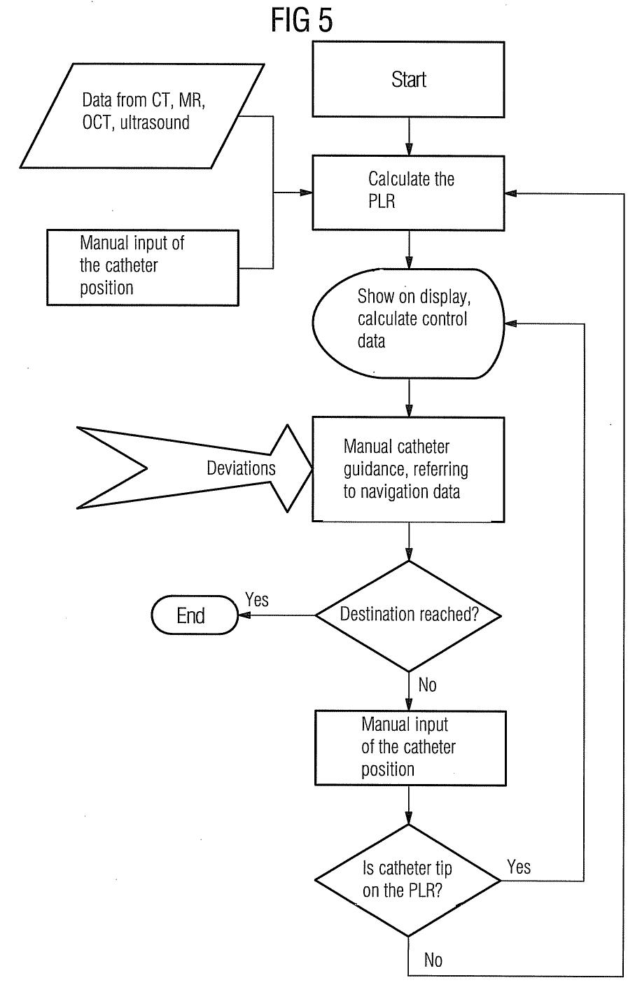

Alternatively, provision can also be made for a semi-automatic mode of operation, supported by the navigation system described above, with manual actuation of the catheter 20 (directly by tactile interaction or indirectly in accordance with a “steer-by-wire” principle), the sequence of activities for which is summarized by way of example in FIG. 5.

1-15. (canceled)

16. A method for navigating a catheter with a catheter tip through a blockage region in a vessel of a patient, comprising:
determining a three-dimensional path through the blockage region;
observing the catheter under a real-time radiological image;
pushing the catheter forward in the vessel along the path;
registering coordinates of the path with the real-time radiological image;
overlaying the path on the real-time radiological image; and
viewing the path by displaying the overlaid image.

17. The method as claimed in claim 16, wherein the real-time radiological image comprises a two-dimensional projection image of the vessel generated by an angiographic X-ray through-illumination method.

18. The method as claimed in claim 16, wherein the path is selected based on a criteria that the catheter tip is advanced along the path with a least possible mechanical resistance.

19. The method as claimed in claim 16, wherein the path is selected based on a criteria that the catheter tip only cuts through blood clots or soft plaque with a high fat content when pushing forward.

20. The method as claimed in claim 16, wherein the catheter tip is automatically kept on the path.

21. The method as claimed in claim 16, wherein the path is determined by a set of sectional images or a 3D representation of the blockage region recorded beforehand in a preliminary investigation.

22. The method as claimed in claim 21, wherein the set of the sectional images or the 3D representation of the blockage region are recorded by a method selected from the group consisting of: computer tomography, 3D angiography, magnetic resonance tomography, 3D ultrasound imaging, positron emission tomography, and single photon emission tomography.

23. The method as claimed in claim 21, wherein breakthrough points for the path are defined in the sectional images of the blockage region and are linked together with each other by a spatial interpolation.

24. The method as claimed in claim 21, wherein a value characteristic of an expected mechanical resistance while pushing forward the catheter is deduced from the sectional images or the 3D representation of the blockage region and is displayed.

25. The method as claimed in claim 21, wherein one of the sectional images of the blockage region corresponding to a current point of the catheter tip in the vessel is displayed in addition to the real-time radiological image.

26. The method as claimed in claim 25, wherein the current position of the catheter tip is detected by a position sensor and is marked on the one of the sectional image that is displayed.

27. The method as claimed in claim 25, wherein a breakthrough point for the path is determined in the one of the sectional images and is marked on the one of the sectional image that is displayed.

28. The method as claimed in claim 25, wherein the current position of the catheter tip is compared with the coordinates of the path.

29. The method as claimed in claim 28, wherein a control signal is generated based on the comparison and is communicated to a steering and drive device that pushes the catheter.

30. The method as claimed in claim 29, wherein the catheter tip is steered by an externally applied magnetic field.

31. The method as claimed in claim 16, wherein data of the blockage region, the path, an actual course of a movement of the catheter, a progress over time of a procedure using the catheter, or a medical and technical equipment used in the procedure is communicated to a database of an associated medical expert system and is stored there.

32. The method as claimed in claim 16, wherein the vessel is a coronary vessel of the patient.

33. A medical investigation and treatment device, comprising:
a catheter that is introduced into a vessel of a patient with a blockage region;
an angiographic imaging system that generates a real-time radiological image for real-time monitoring the catheter while advancing through the blockage region;
an electronic planning unit that:
determines a three-dimensional path through the blockage region,
registers coordinates of the path with the real-time radiological image, and
overlays the path on the real-time angiographic images; and
a display unit that displays the real-time angiographic images with the overlaid path.

34. The medical investigation and treatment device as claim in claim 33, wherein the path is determined by a set of sectional images or a 3D representation of the blockage region recorded beforehand in a preliminary investigation.

35. The medical investigation and treatment device as claimed in claim 33, wherein the catheter is automatically guided on the path by a steering and drive device signaling linked to a targeting guidance unit.
